(19) United States
(12) Patent Application Publication
(10) Pub. No.: US 2024/0085396 A1
(43) Pub. Date: Mar. 14, 2024

(54) Self-Lubricating Soil Probe

(71) Applicant: Cole Cashon, Dallas, TX (US)

(72) Inventor: Cole Cashon, Dallas, TX (US)

(21) Appl. No.: 17/941,226

(22) Filed: Sep. 9, 2022

(51) Int. Cl. G01N 33/24

(52) U.S. Cl. CPC G01N 33/24

(57) ABSTRACT

The present relates generally to a self-lubricating soil probe wherein the probe carries a fluid filled reservoir capable of being engaged such that said fluid flows down the soil probe meeting the group thereby dampening the soil or ground as the probe is pressed into the ground.

### CROSS-REFERENCE TO RELATED APPLICATIONS

None.

### TECHNICAL FIELD

The present invention relates to a lubrication system for soil probes.

### BACKGROUND

The soil probe or auger is the most desirable tool for collecting soil samples or probing beneath the surface. The soil probe works better than a shovel or trowel because it equally collects soil in a continuous core from the surface through the entire sampling depth with minimal disturbance of the soil. It also allows for faster sampling. Some soil collecting tools and probes are used to collect small diameter samples at or near the ground surface, other go deeper into the ground where penetration resistance in high. They allow you to provide a quick soil profile or description, and to determine soil moisture content. Samples may also be collected directly into a liner with some models to allow for later chemical analysis of the soil sample. There are various types of tools known in the industry of soil probes, some collect sand, sludge, and sediment and can recover core samples in a variety of materials including dry sand, loose sediment, and sludge in various mediums. Soil probes are also ideal for locating buried pipes, underground tanks or utility lines. Where the soil probe collects soil or simply probes the soil, the present invention is capable of making such soil probe work more effectively within any ground medium such as dry sand, loose sediment, hard sediment, soil and sludge by increasing fluid mobility in and through those ground mediums.

Soil probes are generally pushed into the soil vertically by hand. They will easily penetrate saturated soils, but not dry soils. The present invention selectively or continuously lubricates the soil probe shaft as it penetrates the ground. The resistance rises as the soil's moisture content falls. The present invention reduces the resistance by maintaining moisture content while probing.

### OBJECTS OF THE INVENTION

A primary object of the present invention is to provide optimal lubrication to a soil probe on the outside of probe rod.

It is another object of the present invention to deepen the depth in which a soil probe can sample and collect in dry soils.

It is another object of the present invention to allow soil probe users to adjust water flow around the probe by a self-adjusting means of controlling water flow.

It is another object of the present invention to fit a soil probe with a refillable water tank for use in the field.

### SUMMARY OF THE INVENTION

The present invention generally discloses a lubricating soil probe consisting of a reservoir capable of holding a liquid (FIG. 1.2), a stable means for connecting the reservoir onto the soil probe between the handle and grips (FIG. 2.2, FIG. 2.4), a sleeve within the housing surrounding the soil probe (FIG. 2.8) with a stopper (FIG. 2.9) on the distal end of the sleeve for preventing a liquid from releasing from the reservoir, and a handle (FIG. 5.5) for operatively releasing the contents of the reservoir onto the probe (FIG. 5.7) by tensioning a spring (FIG. 5.10) thereby lowering the sleeve such that the stopper (FIG. 5.9) lowers thereby allowing liquid to flow down the probe shaft onto the probe.

In one embodiment, the soil probe has a mounted reservoir with a cap (FIG. 1.1) for refilling the reservoir with a liquid, spring(s) (FIG. 2.10) attached to a lever (FIG. 2.5) capable of increasing tension on the springs thereby lowering the sleeve (FIG. 2.8) causing the stopper (FIG. 2.9) to disengage with the resource, the sleeve disposed around to probe and capable of moving up and down with the tension of the spring causing the stopper to lower away from the reservoir thereby releasing the liquid contained therein around the shaft of the probe. The sleeve disposed around to probe is capable of moving up and down with the tension of one or more springs or rubber bands or other similar actuating means. The soil probe can optionally have a hollow portion at its base for receiving a soil sample or can be without such receiving portion.

Other features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific processes disclosed herein. The description of a process by a numeral in a drawing is applicable to the description of that process step shown by that same numeral in any subsequent drawing herein.

FIG. 1 shows the housing for the liquid mounted on the soil probe with an operative handle for releasing such liquid adjacent to a soil probe handle;

FIG. 2 shows a means of mounting the reservoir onto the probe, and a of means connecting the operative level mechanism for releasing the reservoir liquid onto the probe body;

FIG. 3 shows the internal assembly of the present invention;

FIG. 4 shows an alternative view of the internal assembly of the present invention; and

FIG. 5 shows the actuation of the handle lowering the sleeve causing the liquid contained within the housing to begin to flow.

### SPECIFICATION

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The disclosed embodiment provides a preferred embodiment of an invention for lubricating a soil probe.

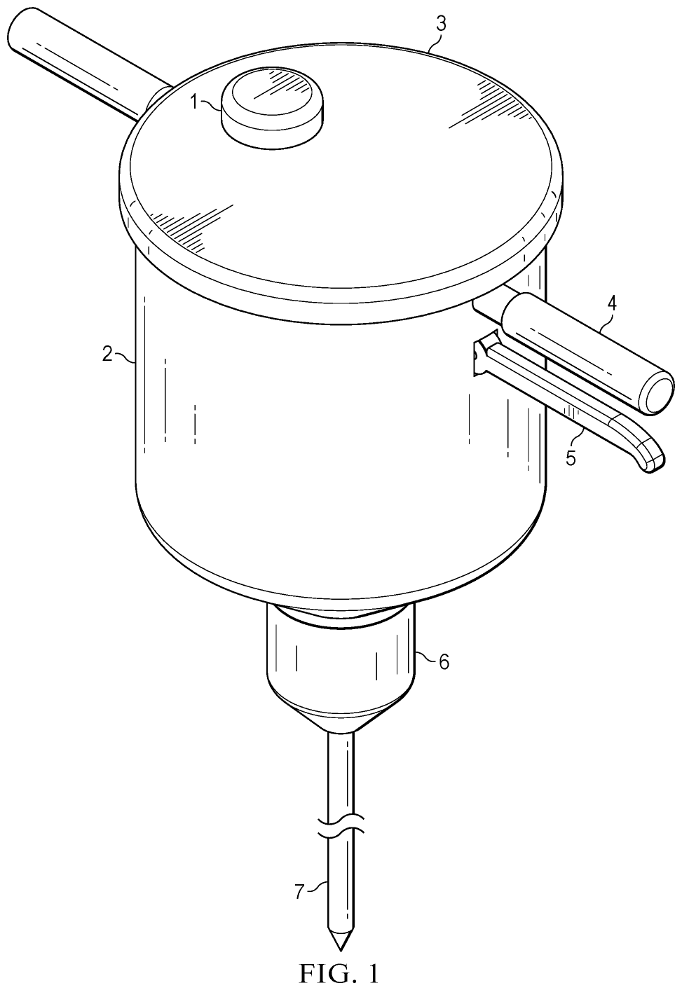

FIG. 1 illustrates components and embodiment of the present invention. 1.1 is a cap used to refill the reservoir 1.2 attached to the probe 1.7. 1.6 is a housing containing an area in which the reservoir will supportively sit, such housing allows space for the stopper to ebb and flow the amount of water delivered to the probe by the handle 1.5 is actuated. 1.3 is an optional removable lid of the entire reservoir top. 1.4 is a handle of the probe.

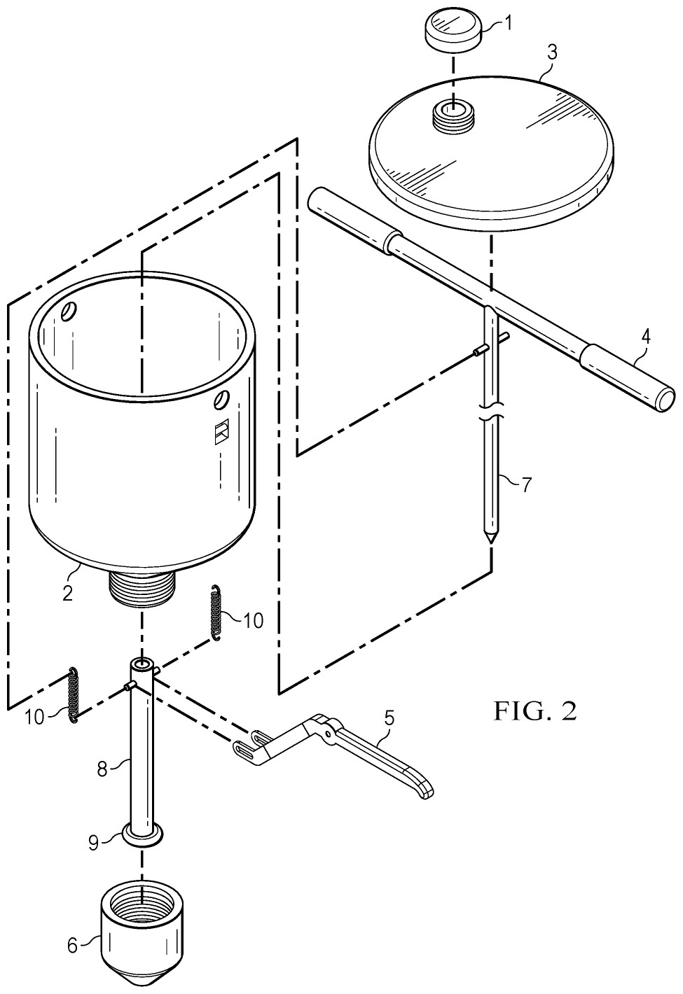

FIG. 2 illustrates components an embodiment of the present invention. 2.1 is a cap used to refill the reservoir 2.2 attached to the probe 2.7. 2.6 is a housing containing an area in which the reservoir will supportively sit, such housing allows space for the stopper to ebb and flow the amount of water delivered to the probe by the handle 2.5 is actuated. 2.3 is an optional removable lid of the entire reservoir top 2.4 is a handle of the probe. Operatively, one or more springs 2.10 or bands are disposed on either side of the shaft 2.8 for actuating the stopper 2.9 up and down when the releasing mechanism is actuated. In this embodiment the releasing mechanism is a handle 2.5, the releasing mechanism could also be a switch or a button if a powered version were utilized.

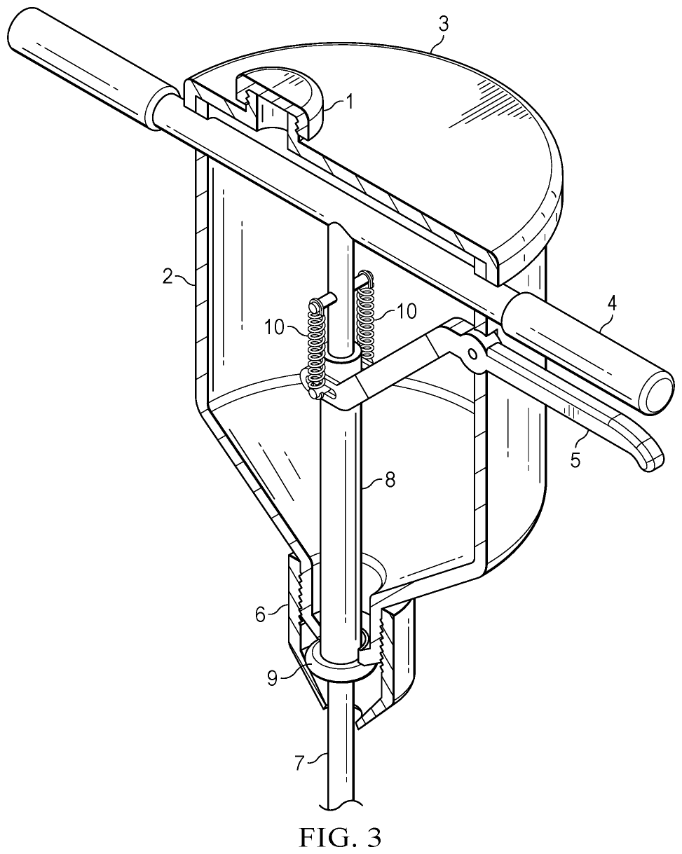

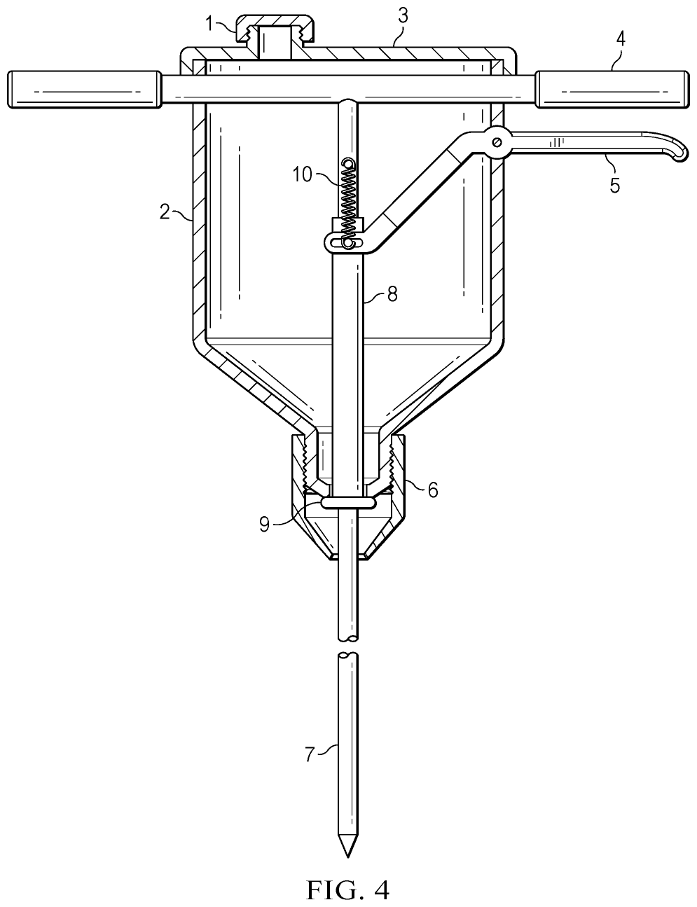

FIG. 3 and FIG. 4 illustrate components and embodiment of the present invention. 3.1 is a cap used to refill the reservoir 3.2 attached to the probe 3.7. 3.6 is a housing containing an area in which the reservoir will supportively sit, such housing allows space for the stopper to ebb and flow the amount of water delivered to the probe by the handle 3.5 is actuated. 3.3 is an optional removable lid of the entire reservoir top 3.4 is a handle of the probe. Operatively, one or more springs 3.10 or bands are disposed on either side of the shaft 3.8 for actuating the stopper 3.9 up and down when the releasing mechanism is actuated. In this embodiment the releasing mechanism is a handle 3.5, the releasing mechanism could also be a switch or a button if a powered version were utilized.

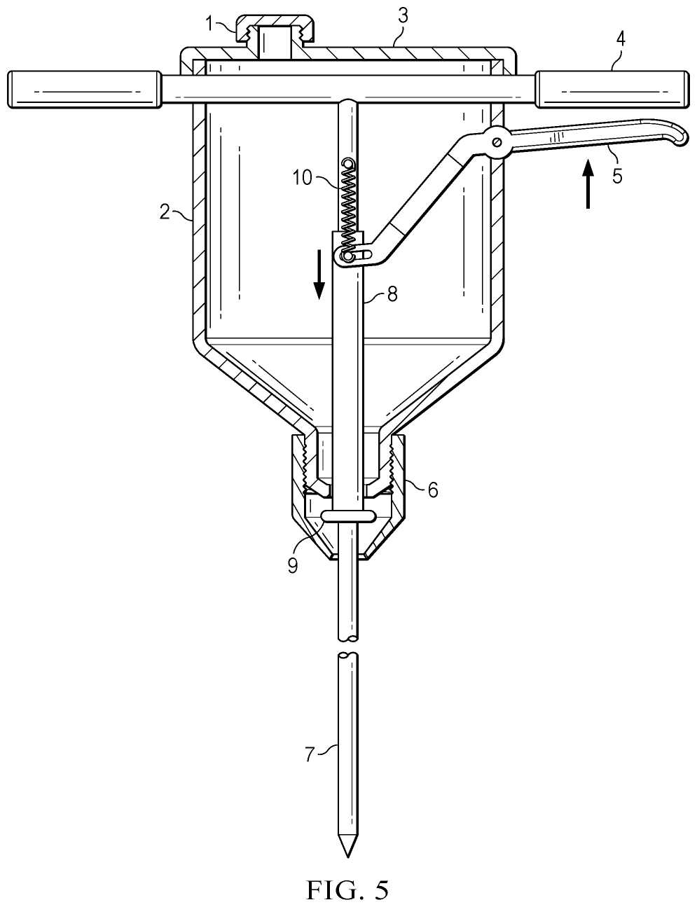

FIG. 5 illustrates the engaged handle of the presentation invention causing an adjustable sleeve to shift downward thereby lowering the stopper such that fluid from the reservoir will flow down onto the probe disposed, in part, within said reservoir. Releases the handle places the invention back in its ready state, or non-flowing state, as shown by FIG. 4.

Preferred embodiments of this invention are described herein, including the best mode known to the inventors for carrying out the invention. It should be understood that the illustrated embodiments are exemplary only and should not be taken as limiting the scope of the invention.

The foregoing description comprises illustrative embodiments of the present invention. Having thus described exemplary embodiments of the present invention, it should be noted by those skilled in the art that the within disclosures are exemplary only, and that various other alternatives, adaptions, and modifications may be made within the scope of the present invention. Merely listing or numbering the steps of a method in a certain order does not constitute any limitation on the order of the steps of that method.

Many modifications and other embodiments of the invention will come to mind to one skilled in the art to which this invention pertains having the benefit of the teachings in the foregoing descriptions. Although specific terms may be employed herein, they are used only in a generic and descriptive sense and not for purposes of limitation. Accordingly, the present invention is not limited to the specific embodiments illustrated herein.

1. A soil probe, comprising:
a hollow portion for receiving a soil sample;
a reservoir for holding a liquid mounted on said soil probe;
said soil probe disposed, in part, within said reservoir; and
a means of releasing said liquid from said reservoir down the body of said soil probe towards the ground.

2. A soil probe, comprising:
a hollow portion for receiving a soil sample;
a reservoir for holding a liquid mounted on said soil probe;
said soil probe disposed, in part, within said reservoir;
an adjustable sleeve surrounding said soil probe disposed within said reservoir;
a stopper surrounding to the distal end of said adjustable sleeve to prevent the release of said liquid; and
a releasing mechanism operatively connected to said adjustable sleeve for releasing said liquid from said reservoir down the body of said soil probe towards the ground.

3. A soil probe, comprising:
a reservoir for holding a liquid mounted on said soil probe;
said soil probe disposed, in part, within said reservoir; and
a means of releasing said liquid from said reservoir down the body of said soil probe towards the ground.

4. A soil probe, comprising:
a reservoir for holding a liquid mounted on said soil probe;
said soil probe disposed, in part, within said reservoir;
an adjustable sleeve surrounding said soil probe disposed within said reservoir;
a stopper surrounding to the distal end of said adjustable sleeve to prevent the release of said liquid; and
a releasing mechanism operatively connected to said adjustable sleeve for releasing said liquid from said reservoir down the body of said soil probe towards the ground.
